(19) United States
(12) Patent Application Publication
(10) Pub. No.: US 2022/0249738 A1
(43) Pub. Date: Aug. 11, 2022

(54) 3D Bioprinted Skin Tissue Model

(71) Applicant: Cellink AB, Göteborg (SE)

(72) Inventors: Isabella Bondesson, Mölndal (SE); Adel Itedal Namro Redwan, Skepplanda (SE); Hector Martinez, Göteborg (SE); Erik Gatenholm, Göteborg (SE)

(21) Appl. No.: 17/617,495

(22) PCT Filed: Jun. 15, 2020
(86) PCT No.: PCT/EP2020/066454

§ 371 Date: Dec. 8, 2021

(30) Foreign Application Priority Data
Jun. 13, 2019 (SE) 1950711-0

(51) Int. Cl. A61L 27/20; A61L 27/38; A61L 27/44; A61L 27/60; C12N 5/00; C12N 5/071

(52) U.S. Cl. CPC A61L 27/20; A61L 27/3804; A61L 27/44; A61L 27/60; C12N 5/0062; C12N 2533/74; C12N 2533/54; C12N 2503/02; C12N 2503/06; C12N 2513/00; C12N 2533/52; C12N 5/0698

(57) ABSTRACT

The present invention relates to a 3D bioprinted skin tissue model, a method for providing said model and the use of said model. The 3D bioprinted skin tissue model comprises at least one bioink A, at least one cell type A, at least one factor A, wherein the bioink A comprises at least one biopolymer, a thickener, at least one extra-cellular matrix or a decellularized matrix, and optionally a photo initiator and/or cellular additions, the at least one cell type A is an epidermal, dermal and/or hypodermal cell or cell line, and the at least one factor A is a growth factor, protein and/or molecule that stimulates altered or abnormal metabolism of cell type A.

### TECHNICAL FIELD

The present invention relates to the field of 3D bioprinting of tissue, and in particular to skin tissue models.

### BACKGROUND ART

In the field of in vitro models for skin, the gold standard for cell culture is cells grown in two-dimensional (2D) culture that are allowed to form confluent layers mimicking the epidermal compartment. This 2D culture is preformed either on tissue culture plastic, potentially covered with a feeder layer of dermal fibroblasts, or on a three-dimensional (3D) construction with dermal fibroblasts. This 3D construction is normally molded, or a scaffold seeded with dermal fibroblasts. Since cells within the human body are organized and distributed in 3D space, these types of cell cultures have enhanced similarities with native tissue environment. However, these methods are labour intense and do not allow for controlled construction of the in vitro models. The development of standardized models that can provide highly relevant information of cell and human physiology for research is therefore needed.

WO2018064778 A1 discloses a handheld device for bioprinting biomaterials and tissues. For printing, a solution is used that is a mixture of natural or synthetic biopolymer solution with cells and/or growth factors. It is disclosed that a hydrogel, non-cell containing layer, is first printed as a mechanical support for the layers including cells.

### SUMMARY OF THE INVENTION

3D bioprinted skin tissue models, which allow for automated production of in vitro skin tissue models with controlled deposition of cells, biomaterials and biomolecules contributes to the advancement of the field of skin research. A 3D skin tissue model is of high interest for applications in drug development, compound testing, rejuvenation research, regenerative tissue engineering research, tissue engineering, photosensitivity testing, drug and/or molecular compound absorption testing, toxicology studies, irritant studies, allergen testing and regenerative medicine for both physiological, defect and pathological understanding due to the skins important dual function as protection and interface towards external environments, both for the internal and external skin linings of the body, such as the skin, the oesophagus and the urethra. A 3D skin tissue model with highly relevant human physiological mimicry will improve efficiency of therapeutic, biological and skin care product development and research.

Thus, it is an object of the present invention to provide a 3D skin tissue model with a controlled construction. It is also an object to provide a method for producing a 3D skin tissue model, said method being effective, easy and enabling control of the construction of the skin tissue model.

Furthermore, it is an object of the present invention to provide a 3D skin tissue model that allow for bioprinting directly onto the print surface (such as plastics or glass) in vitro.

Still further, it is an object of the invention to provide a 3D skin tissue model that enables the production of several replicates, which when produced have low or minimal variation.

Moreover, it is an object of the present invention to provide a 3D skin tissue model that allows for dispensing at a great variety of sizes and resolution, thereby allowing e.g. a broad range of models and/or types of models of different sizes, and within different container sizes and/or on top of different surfaces.

The objects are attained in a first aspect by a method of producing a skin tissue model in an automated manner, a necessity to achieve a robust production of skin tissue models for drug screening or chemical testing in a standardized manner, for example, comprising the steps of:

(a) providing at least one bioink A;
(b) providing at least one cell type A;
(c) providing at least one factor A;
(d) mixing the components provided in steps (a)-(c), and optionally other components, in such proportions that allows printability for the mixture, and that provides a viable setting for the at least one cell type A;
(e) bioprinting and/or dispensing the resulting mixture in an automated and reproducible manner, whereby a tissue model is formed, said tissue being characterized as a skin tissue;
wherein the bioink A comprises at least one biopolymer such as collagen, methacrylate collagen (ColMA), gelatine, methacrylate gelatine (GelMA), cellulose, nano-fibrillar cellulose, alginate, chitosan, acacia gum, tara gum, glucomannan, pectin, locust bean gum, guar gum, carrageenan or tragacanth, a thickener such as nanocellulose, glucomannan, xanthan gum, gellan gum, diutan gum, welan gum, pullalun gum, collagen or gelatin, at least one extra-cellular matrix or a decellularized matrix component such as glycoamino glycans, collagens, elastin, proteoglycans, aggrecans, isolated laminins, glycol-amino-glycans such as hyaluronic acid and heparin, purified molecular proteins such as fibrinogen and fibrin and/or purified molecular proteins motifs such as RGD-motif, and optionally a photo initiator, such as Lithium phenyl-2,4,6-trimethylbenzoylphosphinate (LAP) or irgacure, and/or cellular additions;
wherein the thickener is a polysaccharide-based substance, such as nanocellulose, glucomannan, xanthan gum, gellan gum, diutan gum, welan gum or pullalun gum, or a protein-based substance, such as collagen or gelatin, that modulates the viscosity of the bioink A;
the at least one cell type A is an epidermal, dermal and/or hypodermal cell or cell line of human and/or animal origin, said cells optionally being primary cells, immortalized and iPSC- or ESC-derived, such as keratinocytes, melanocytes, fibroblasts, sebocytes, dendritic cells, macrophages, stem cells, induced pluripotent stem cells, adipocytes, glandular cells or follicle cells;
and the at least one factor A is a protein or molecule, that will stimulate altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition, and/or a growth factor, such as fibroblast growth factor (FGF), epidermal growth factor (EGF), or vascular endothelial growth factor (VEGF), and/or small molecules, macro molecules, and/or proteins such as cytokines, hormones, lipids, carbohydrates or nucleic acids, that stimulates altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition.

In embodiments, the following quantities of components are used:

the bioink A comprises (based on total weight of the bioink) 2-15% w/w, preferably 2-10% w/w, of at least one biopolymer, 0.5-3% w/w of thickener, 0.1-2% w/w of at least one extra-cellular matrix or a decellularized matrix component, and optionally 0.05-1% w/w of a photo initiator and/or 1×10²-1×10⁷ cellular additions per ml;
the at least one cell type A is used in quantities of 1×10³-10×10⁷ cells per 1 mL Bioink and/or 1×10³-10×10⁵ cells per 1 cm²;
the at least one factor A is used in quantities of 1×10⁻⁹-1×10⁻³ molar for growth factors, such as fibroblast growth factor (FGF), epidermal growth factor (EGF), keratinocyte growth factor (KGF), vascular endothelial growth factor (VEGF), and 1×10⁻⁶-1×10⁻¹ molar and/or 1-1000 mg/mL of other factors, such as glycosaminoglycans, collagens, elastin, proteoglycans, aggrecans, isolated laminins, glycol-amino-glycans such as hyaluronic acid and heparin, purified molecular proteins such as fibrinogen and fibrin and/or purified molecular proteins motifs such as the RGD-motif, cytokines, hormones, lipids, carbohydrates or nucleic acids that stimulates altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition.

Hereby, the objects of the invention are accomplished. More specifically, for example the skin tissue model of the invention can be dispensed at different levels of resolution (6-34 G nozzle/needle or 10-0.01 mm), where all sizes/resolution levels can be utilized within one model or used to create different types of models. Allowing for adaptation of the model to different interests, such as drug testing, cosmetic testing, disease modelling or cell signalling research, analysis set ups and/or customized to fit in different bioreactors and/or prefusion systems. Any bioprinter of choice can be used, and several replicates of the skin tissue model can be made, which when bioprinted has low variation and controlled deposition of cells and materials. Also, the present invention enables the models to be bioprinted at different sizes and printed within and/or on top of different container sizes, such as petri dishes, chips, slides, vascularized modules, well plates and transwells of different sizes, and on top of different surfaces, such as glass, plastics (treated and non-treated), biomaterials, coatings and/or polymers of different kind.

Moreover, for creating the skin tissue model of the invention, precise deposition of the materials are needed/intended as tool. A requirement will be to use nozzle/needles and a bioprinter that precisely can dispense the material at the right location within the model. The bioink compositions with thickeners according to the invention allow for production of stand-alone, robust in-vitro models that after deposition retain its shape, before and after curing. A pre-request to shape the different types of 3D in-vitro model designs.

According to one embodiment, at least one additional cell type A, at least one additional bioink A, and at least one additional Factor A is provided, wherein the two or more bioinks are formulated so that bioink A supports one cell type A and the additional bioink(s) A support(s) a second or further cell type A.

According to one embodiment, the method further comprises step (f) of providing a cell suspension A, and applying said cell suspension A to the tissue formed in step (e).

According to one embodiment the cell suspension A comprises of cell relevant medium and/or materials synthetically derived or derived from bacteria, plants and/or animals, such as gelatine methacrylate, collagen, collagen methacrylate, alginate or cellulose, optionally a thickener, a cell type A, factors specific to cell type A which are proteins or molecules that will stimulate altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition, optionally a photo initiator, optionally extracellular matrix proteins. This since the response of cells within the 3D bioprinted model is highly dependent on the matrix in the bioink as well as the influx of signals during the culture to, for example, achieve different phenotypes and/or reach full maturation.

According to one embodiment, the at least one biopolymer is chosen from the group comprising a nanocellulose or nanofibrillar cellulose, a gelatine, such as gelatine methacrylate, collagen, such as collagen methacrylate, alginate, acacia gum, tara gum, glucomannan, pectin, locust bean gum, guar gum, carrageenan, and tragacanth.

According to one embodiment, the thickener is a polysaccharide- or protein-based substance that modulates the viscosity of the bioink A to the degree that allows for reproducible bioprinting and/or dispensing. Polysaccharide-based thickeners may include nanocellulose, glucomannan, xanthan gum, gellan gum, diutan gum, welan gum, and pullalun gum, whereas protein-based thickeners may include collagen and gelatine.

According to one embodiment, the extra-cellular matrix or decellularized matrix components originate from a human or animal source, and may be chosen from the group comprising of glycosaminoglycans, collagens, elastin, proteoglycans, aggrecans, isolated laminins, glycol-amino-glycans such as hyaluronic acid and heparin, purified molecular proteins such as fibrinogen and fibrin and/or purified molecular proteins motifs such as the RGD-motif.

The photoinitiator is required to start the photocrosslinking process of the bioink when light of specific wavelength range is applied. According to one embodiment, the photoinitiator can be chosen from lithium phenyl-2,4,6-trimethylbenzoylphosphinate (LAP) or Irgacure. LAP is preferred for more sensitive cells as it can be used at a higher frequency range above 400 nm wavelength, which is less detrimental to cells. Irgacure is a more powerful photoinitiator requiring shorter time of photocrosslinking and resulting in stiffer constructs, however it can be excited only with light wavelength around 350 nm that cannot be tolerated by all cells.

According to one embodiment, the cellular addition is chosen from one or more of sebocytes, glandular cells, and/or follicle cells. The cellular additions advance the complexity of the bioprinted skin tissue model to mimic the native skin tissue to larger extent. To fully replicate native skin tissue and the native cross-talk within the native skin tissue all cell types and cellular appendages of the skin, such as hair follicles, sebaceous glands and sweat glands, need to be represented within the model.

According to one embodiment, the one or more cell type A is/are chosen from:

(i) Epidermal cells such as keratinocytes, melanocytes, and/or epithelial cells originating from induced pluripotent stem cells, embryonic stem cells, other stem cells, primary cells, and cell lines of human and/or animal origin; or
(ii) Dermal cells such as fibroblasts, endothelial cells, Schwann cells and/or dendritic cells originating from induced pluripotent stem cells, embryonic stem cells, other stem cells, primary cells, and cell lines of human and/or animal origin; or
(iii) Hypodermal cells such as adipose cells, fibroblasts and/or macrophages originating from induced pluripotent stem cells, embryonic stem cells, other stem cells, primary cells, and cell lines of human and/or animal origin.

According to one embodiment, the at least one cell type A originates from macro locations, such as a facial location, breast, belly, urethra, oesophagus and/or head, and/or from micro locations, such as papillary dermis or reticular dermis, of the body of healthy, diseased and/or defected human and/or animal sources.

According to one embodiment, the factor A is a growth factor such as fibroblast growth factor (FGF), epidermal growth factor (EGF), or vascular endothelial growth factor (VEGF) and/or small molecules, macro molecules, and/or proteins such as cytokines, hormones, lipids, carbohydrates or nucleic acids, that stimulates altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition.

According to one embodiment, step (e) is performed using an extrusion, syringe or ink-jet based bioprinting device.

According to one embodiment, the tissue is bioprinted or dispensed in a manner that produces two or more compartments and/or one or more cellular gradient(s) within the tissue.

According to one embodiment, the tissue is bioprinted or dispensed to form a hypodermal compartment, a dermal compartment and/or an epidermal compartment, and optionally a cellular and/or molecular gradient is bioprinted or dispensed within one or more compartment(s). With gradient a difference in cellular, additive molecules, proteins, growth factors such as fibroblast growth factor (FGF), epidermal growth factor (EGF), keratinocyte growth factor (KGF), vascular endothelial growth factor (VEGF), glycosaminoglycans, collagens, elastin, proteoglycans, aggrecans, isolated laminins, glycol-amino-glycans such as hyaluronic acid and heparin, purified molecular proteins such as fibrinogen and fibrin and/or purified molecular proteins motifs such as the RGD-motif, cytokines, hormones, lipids, carbohydrates and/or nucleic acids concentration is bioprinted or dispensed in different layers or locations within the model. This will enable mimicking of natural gradients found within native skin tissue and enhance the relevance and complexity of the bioprinted skin tissue model. Making it more similar to native skin tissue and provide more relevant and natural response of embedded cells.

According to one embodiment, the two or more compartments and/or the cellular gradient(s) are bioprinted or deposited at the same occasion and/or at one or more later occasions.

According to one embodiment, the Factor A is chemically attached to, or trapped in, the at least one Bioink A and/or additional bioinks A, and/or incorporated with the at least one cell type A.

According to one embodiment, the produced skin tissue model is further subject to a culturing method wherein the skin tissue model is cultured

(i) by being submerged in medium;
(ii) in a flow device to mimic a vascular system; and/or
(iii) at an air-liquid interface.

According to one embodiment, a combination of one or several culturing methods is used for the same skin tissue model, either simultaneously and/or sequentially.

According to a second aspect, a 3D bioprinted skin tissue model is provided, comprising

i. at least one bioink A
ii. at least one cell type A
iii. at least one factor A
wherein the bioink A comprises at least one biopolymer, a thickener, at least one extra-cellular matrix or a decellularized matrix, and optionally a photo initiator and/or cellular additions; the at least one cell type A is an epidermal, dermal and/or hypodermal cell or cell line of human and/or animal origin, said cells optionally being primary cells, immortalized and iPSC- or ESC-derived;
wherein the thickener is a polysaccharide-based substance, such as nanocellulose, glucomannan, xanthan gum, gellan gum, diutan gum, welan gum or pullalun gum, or a protein-based substance, such as collagen or gelatin;
the at least one factor A is a protein or molecule that will stimulate altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition, and/or a growth factor, such as fibroblast growth factor (FGF), epidermal growth factor (EGF), or vascular endothelial growth factor (VEGF), and/or small molecules, macro molecules, and/or proteins such as cytokines, hormones, lipids, carbohydrates or nucleic acids that stimulates altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition. According to embodiments:
the bioink A comprises (based on total weight of the bioink) 2-15% w/w, preferably 2-10% w/w, of at least one biopolymer, 0.5-3% w/w of thickener, 0.1-2% w/w of at least one extra-cellular matrix or a decellularized matrix component, and optionally 0.05-1% w/w of a photo initiator and/or 1×10²-1×10⁷ cellular additions per ml;
the at least one cell type A is used in quantities of 1×10³-10×10⁷ cells per 1 mL Bioink and/or 1×10³-10×10⁵ cells per 1 cm²; and/or
the at least one factor A is used in quantities of 1×10⁻⁹-1×10⁻³ molar for growth factors, and 1×10⁻⁶-1×10⁻¹ molar and/or 1-1000 mg/mL of other factors.

According to one embodiment, the at least one biopolymer is chosen from a collagen, methacrylate collagen (ColMA), gelatine, methacrylate gelatine (GelMA), cellulose, nano-fibrillar cellulose, alginate, chitosan, acacia gum, tara gum, glucomannan, pectin, locust bean gum, guar gum, carrageenan or tragacanth.

According to one embodiment, the model further comprises an additional cell suspension A, said cell suspension A comprises of cell relevant medium and/or materials synthetically derived or derived from bacteria, plants and/or animals, such as gelatine methacrylate, collagen, collagen methacrylate, alginate or cellulose, optionally a thickener, a cell type A, factors specific to cell type A which are proteins or molecules that will stimulate altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition, optionally a photo initiator, optionally extracellular matrix proteins.

According to one embodiment, the one or more cell type A is/are chosen from

(i) Epidermal cells such as keratinocytes, melanocytes, and/or epithelial cells originating from induced pluripotent stem cells, embryonic stem cells, other stem cells, primary cells, and cell lines of human and/or animal origin; or
(ii) Dermal cells such as fibroblasts, endothelial cells, Schwann cells and/or dendritic cells originating from induced pluripotent stem cells, embryonic stem cells, other stem cells, primary cells, and cell lines of human and/or animal origin; or
(iii) Hypodermal cells such as adipose cells, fibroblasts and/or macrophages originating from induced pluripotent stem cells, embryonic stem cells, other stem cells, primary cells, and cell lines of human and/or animal origin.

According to one embodiment, the model comprises at least one compartment, representing a hypodermal, a dermal and/or an epidermal compartment.

According to one embodiment, the model comprises two or more compartments representing a biological gradient corresponding to a hypodermal, a dermal and/or an epidermal compartment; and optionally comprising a biological gradient within one or more of said compartments.

According to a third aspect, the use of a 3D bioprinted skin tissue model according to the above is provided, in one or more of:

(i) Developmental biology in order to gain understanding of cellular activities within a 3D environment such as cellular distribution, migration, proliferation, matrix production, interactions with other cells and the surrounding environment, etc.; and/or
(ii) Compound testing for cosmetic and skin care product evaluation, toxicity studies, irritant studies, allergen testing, metabolism studies, tissue and/or cellular rejuvenation investigations, photosensitivity testing, drug and/or molecular compound absorption testing, cellular differentiation/maturation, spheroid differentiation/maturation, organoid differentiation/maturation, etc.; and/or
(iii) Tissue regeneration and rejuvenation applications such as tissue remodelling, cellular proliferation, cellular metabolism, cellular differentiation/maturation, cell-cell interaction, cell-matrix interaction, cellular crosstalk/signalling, vascularization, etc.; and/or (iv) Pharmaceutical applications for drug discovery, target validation, allergen studies, toxicity studies, metabolism studies, cellular differentiation/maturation, spheroid differentiation/maturation, organoid differentiation/maturation, etc.; and/or
(v) Medical device evaluation and development, toxicity studies, allergen studies etc. for devices in contact with internal and/or external skin linings; and/or
(vi) Stem cell research with focus on cellular differentiation and maturation as dispersed cells, spheroids, organoids, etc.

According to one embodiment, the use of the 3D bioprinted skin tissue model according to the above is in applications relating to both internal and external skin linings such as the skin, oesophagus and urethra.

### BRIEF DESCRIPTION OF THE DRAWINGS

FIG. 1: Illustration of different compartments that can be used to build and design a bioprinted skin tissue model. The different illustrations show example of block compartments (A), brim compartments (B), rigid compartments (C), a combination of block and brim compartments (A+B) and a combination of block and rigid compartments (A+C).

FIG. 2: Blueprint of how the model could be composed. A is the epidermal compartment with a high concentration of epidermal cells (the triangles). B and C represents the dermal compartment with a biological gradient of dermal cells (the stars), with a higher concentration in the part towards the epidermal compartment (B) and a lower concentration in the bottom part (C).

FIG. 3: Effect of human dermal fibroblasts cultured in one composition of bioink at day 14 with (B) or without (A) epidermis. Figure B shows elongated morphology typical for dermal fibroblasts. Figure C show deposition of different compartments within the construct, with brighter cells representing the epidermis and less bright cells the dermis. Image A and B is in 10× magnification, image C is in 4× magnification.

FIG. 4: Effect on elastin production within a skin tissue model as response to treatments. In control sample, being the basic bioprinted full thickness skin tissue model, there is low expression of elastin while in the treated model there is a sharp increase of elastin production. The collagen type 1 production is not affected. Images are captured in 10× magnification, scale bar is 100 μm.

FIG. 5: Example of cellular response of the skin model to different treatments in one composition of bioink at day 14 (A, C) and day 28 (B, D). Images show collagen type I expression in non-treated model (A-B) and model treated with a biomolecule (C-D). Images are captured at 10× magnification.

FIG. 6: H&E staining of crossection of 3D bioprinted skin tissue model in which the epidermis (light gray) is forming on top of the dermis (dark gray). Image captured at 4× magnification, scale bar 200 μm.

FIG. 7: The diagram showing the effect of different thickeners on viscosity (XG—xanthan gum, Glu—glucomannan, NFC—nanofibrillated cellulose).

FIG. 8: Temperature sweeps of GelMA (A) and thickener modified GelXG (B). Gel point for GelMA is indicated as an intercept between storage and loss moduli curves.

FIG. 9: Comparative image of constructs bioprinted with hydrogels containing 1%, 2% and 3% of xanthan gum.

FIG. 10: Flow sweeps of alginate (A) and alginate modified with nanocellulose (CELLINK Bioink, B). Shear thinning behaviour is indicated as the decrease of viscosity with the increase of shear rate.

### DETAILED DESCRIPTION

The present invention relates to a skin tissue model composed of cells, bioinks and biomolecules to be used for scientific research within in the 3D skin tissue-modelling field. The applications for such a tissue model can be specific for cosmetic compound evaluation and/or discoveries, evaluation of medical devices, skin care compound evaluation and/or discoveries, pharmaceutical evaluations and/or discoveries, regenerative medicine investigations, tissue and/or cellular rejuvenation investigations, photosensitivity testing, drug and/or molecular compound absorption testing, tissue engineering developments, toxicology studies, irritant studies, allergen testing and skin physiology and/or pathology. The cells, bioinks and biomolecules may be layered with specific deposition to mimic the native distribution of cells and extra cellular matrix within native skin, both for the internal and external skin linings, such as the skin, the oesophagus and the urethra. When the constructed skin tissue models have been stabilized in culture, the model is cultured at air-liquid interface to mimic native environment of the skin and stimulate differentiation of the epidermis as well as maturation of the model. The model can also be cultured in a flow device to mimic native distribution of nutrients and/or the sporadically flow of body liquids over the internal skin linings.

The skin is an organ with clear, distinct layers with different, specific compositions within the different layers. Therefore, both methods to construct a model with distinct organizations as well as materials that can support the creation of these specific layered models are required to create skin tissue models resembling native tissue. The 3D bioprinting method enables specific deposition of biomaterial with cells and biomolecules, as well as flexibility to tune the concentration of the cells and/or biomolecules within the bioink, the architecture of the construct, the localization of the cells and/or biomolecules and the spatial organization of the cells/bioinks and/or biomolecules. There is a requirement to create models with enhanced mimicry of physiological, pathological and/or defected conditions. The bioinks, being printable mixtures of biomaterials and/or biomolecules, enables the creation of these models as well as making the utilization of bioprinting possible.

The bioinks are tailored to encourage the tissue maturation towards normal, defect or pathological skin function. The bioink is based on either a synthetic and/or natural biopolymer incorporated with extracellular matrix proteins that simulates the skin niche environment. The biopolymer can be a polysaccharide derived from botanical sources such as cellulose of different fibrillar structures, alginate, acacia gum, tara gum, glucomannan, pectin, locust bean gum, guar gum, carrageenan, and tragacanth. The bioink can include thickeners known as biogums such as xanthan gum, gellan gum, diutan gum, welan gum, and pullalun gum or other thickeners such as nanocellulose, glucomannan, collagen or gelatine. Incorporation of extracellular matrix proteins derived from human and/or animal sources such as decellularized extracellular matrices, isolated laminins, glycol-amino-glycans such as hyaluronic acid and heparin, purified molecular proteins such as collagen, elastin, fibrinogen and fibrin, and/or purified molecular proteins motifs such as the RGD-motif. Each component of the bioink is essential for printability, crosslinking, cellular attachment, cellular proliferation, cellular maturation, and cellular functionality. With the balanced niche provided by the composition of the bioink, the skin cells, epidermal, dermal and/or hypodermal cells, of animal or human origin, primary or iPSC- or ESC-derived, will maintain their respective physiological, pathological and/or defect states as directed by the stimulating factors.

The cells used in the skin tissue model are of human, preferably, and/or animal sources isolated from skin tissues or derived from stem cells such as embryonic and/or induced pluripotent stem cells, and models the functionality of epidermal, dermal and/or hypodermal cells within the skin tissue. Cells such as fibroblasts, keratinocytes and melanocytes, mono-cultured or co-cultured in different combinations, are commonly used to investigate cellular effect of compounds in vitro. Keratinocytes are either cultured in 2D to form a compact, confluent layer mimicking the epidermis or seeded on top of a fibroblast feeder layer or structure for permeability, topological absorption of compounds or irritant/toxicology testing. The fibroblast structures are normally a moulded structure of material, commonly collagen or fibrin, mixed with the fibroblast cells or a scaffold seeded with fibroblasts. The structures are normally allowed to maturate before keratinocytes are seeded on top. For a photosensitive model, a heterogeneous mixture of keratinocytes and melanocytes is normally seeded instead of the keratinocytes. All these methods are labour intense and requires several steps of manual handling. By using 3D bioprinting, the 3D skin tissue model generated with the herein described cells, bioinks and biomolecules will require less manual handling to ensure robustness in replicates.

The printability properties of the bioinks enables specific deposition and arrangement of the different cell types and biomolecule components in relation to each other. The composition and architecture can be defined for specific questions. For example, the fibroblasts can be bioprinted in a gradient with layers of high concentration of fibroblast deposit on top of layers with lower concentration of fibroblasts. Alternatively, a specific component can be incorporated in the epidermal compartment and/or layered in the dermal compartment, encapsulating the cell type in question to investigate the cellular effect of the component, the paracrine communication, and/or the functionality of the cells within the tissue model.

The generation of skin models with functionalized bioinks can be layered to mimic epidermal, dermal and hypodermal compartment of native skin and provide functional skin tissue models of both the internal and external skin linings of the body, such as the skin, the oesophagus and the urethra. The bioinks are formulated from synthetically and naturally derived biopolymers, macromolecules, proteins, and small molecules from plants, microbial, animals, and/or human sources. Biopolymers include but not limited to polysaccharides such as cellulose of different fibrillar structures, extracellular matrix proteins derived from animal/human tissues such as glycosaminoglycans, collagens, elastin, proteoglycans, laminins, and aggrecans. The bioinks formulations constitute of other components to enhance printability, viscosity, crosslinking capability, degradation, and cellular metabolism/activity. The bioinks provided will have unique capacities to support the metabolism, proliferation and functionality of the cell types of interest. By functionalizing the bioink with skin specific laminins, skin specific extracellular matrix proteins derived from skin such as animal or human of different conditions such as age, possible disease, extraction methods of the proteins, and other macromolecules such as exosomes, proteins, ligands, factors isolated/extracted from different animal/human tissues, a niche environment is obtained which will support cell lines, stem cells, such as ESCs or iPSCs, cellular additions, such as sebocytes, glandular cells, follicle cells, and primary cells of both animal and human origin.

The employed cells will preferably be of human origin in order to elevate the relevance of the 3D skin tissue model, especially for pre-clinical based applications in order to facilitate the translation to clinical trials and/or simulate human response in order to limit animal testing. These cells can be of human or animal origin, it may be cell lines, primary cells, and heterogeneous mixtures of cells, which are currently utilized in skin research. Cells include but are not limited to primary, immortalized and ESC- or iPSC-derived dermal fibroblasts, commonly utilized to model the dermal compartment of skin and primary, immortalized and ESC- or iPSC-derived keratinocytes, commonly utilized to model the epidermal compartment of skin. The dermal function of fibroblasts is to moderate the composition of the extra cellular matrix composition. The epidermal function of keratinocytes is to provide the skin barrier of the epidermis. Primary, immortalized and ESC- or iPSC-derived melanocytes are commonly utilized to model the photo protective function of the epidermal compartment of skin. Primary, immortalized and ESC- or iPSC-derived adipose cells are utilized in combination with dermal fibroblasts to model the hypodermal compartment of the skin. For elevated human relevance epithelial stem cells, endothelial cells such as human dermal microvascular endothelial cells, Schwann cells, dendritic cells and/or macrophages, primary, immortalized and ESC- or iPSC-derived, and/or cellular additions, such as but not limited to sebocytes, glandular cells and follicle cells, can be incorporated to provide a more complex tissue model.

The skin tissue model of the present disclosure may be used for cosmetic compound evaluation and/or discoveries, evaluation of medical devices, skin care compound evaluation and/or discoveries, pharmaceutical evaluations and/or discoveries, regenerative medicine investigations, tissue and/or cellular rejuvenation investigations, photosensitivity testing, drug and/or molecular compound absorption testing, tissue engineering developments, toxicology studies, irritant studies, allergen testing and skin physiology and/or pathology chemical and/or mechanical stimulants are necessary. Hence, the model needs to be responsive to simulating factors in order to be functional. Common chemical factors used in the field are hyaluronic acid, VEGF, FGF, EGF, KGF, CaCl₂, L-ascorbic acids, and other molecules, which can drive for example, over-production of extracellular matrix by the fibroblast cells, as well as vascularization or enhanced proliferation of the keratinocytes. By the use of for example perfusion culture and flow chambers, mechanical factors are provided to the culture to reproduce the mechanical stress conditions that may be present in native tissue.

The present disclosure thus provides, in a first aspect, for a method of producing a skin tissue model in an automated manner, comprising the steps of:

(a) providing at least one bioink A;
(b) providing at least one cell type A;
(c) providing at least one factor A;
(d) mixing the components of steps (a)-(c), and optionally other components, in such proportions that allows printability for the mixture, and that provides a viable setting for the at least one cell type A;
(e) bioprinting and/or dispensing the resulting mixture in an automated and reproducible manner, whereby a tissue model is formed, said tissue being characterized as a skin tissue.

The bioink A comprises at least one biopolymer, a thickener, at least one extra-cellular matrix or a decellularized matrix component, and optionally a photo initiator and/or cellular additions. Typically, the bioink A comprises (based on total weight of the bioink) 2-15% w/w, preferably 2-10% w/w, of at least one biopolymer, 0.5-3% w/w of thickener, 0.1-2% w/w of at least one extra-cellular matrix or a decellularized matrix component, and optionally 0.05-1% w/w of a photo initiator and/or 1×10²-1×10⁷ cellular additions per ml.

The at least one cell type A is an epidermal, dermal and/or hypodermal cell or cell line of human and/or animal origin, said cells optionally being primary cells, immortalized and iPSC- or ESC-derived. Typically, the at least one cell type A is used in quantities of 1×10³-10×10⁷ cells per 1 mL Bioink and/or 1×10³-10×10⁵ cells per 1 cm².

The at least one factor A is a growth factor such as fibroblast growth factor (FGF), epidermal growth factor (EGF), or vascular endothelial growth factor (VEGF) and/or small molecules, macro molecules, and/or proteins such as cytokines, hormones, lipids, carbohydrates or nucleic acids, that stimulates altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition. Typically, the at least one factor A is used in quantities of 1×10⁻⁹-1×10⁻³ molar for growth factors, and 1×10⁻⁶-1×10⁻¹ molar and/or 1-1000 mg/mL of other factors, such as proteins or molecules.

Step (e) is preferably performed using an extrusion, syringe or ink-jet based bioprinting device.

According to one embodiment, at least one additional cell type A, at least one additional bioink A, and at least one additional Factor A is provided in the method. The two or more bioinks present are preferably formulated so that bioink A supports one cell type A and the additional bioink(s) A support(s) a second or further cell type A. For instance, if three bioinks are provided, and three cell types A, the first bioink A will support the first cell type A, the second bioink A will support the second cell type A, and the third bioink A supports the third cell type A. It is however also possible that two bioinks are provided that support the same cell type A, but with a different formulation so at to regulate or control the cellular development for cell type A in different manners. Moreover, it is also possible that one bioink support more than one cell type, such as a first and a second, and also a third or more, cell type A.

According to one embodiment, the method according to the first aspect further comprises a step (f) of providing a cell suspension A, and applying said cell suspension A to the tissue formed in step (e). The cell suspension A comprises of cell relevant medium and/or materials synthetically derived or derived from bacteria, plants and/or animals, such as gelatine methacrylate, collagen, collagen methacrylate, alginate or cellulose, optionally a thickener, a cell type A, factors specific to cell type A which are proteins or molecules that will stimulate altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition, optionally a photo initiator, optionally extracellular matrix proteins.

The biopolymer used in the bioink is chosen from a nanocellulose or nanofibrillar cellulose, or a gelatine, such as gelatine methacrylate, or a collagen. The biopolymer can be a polysaccharide derived from botanical sources such as acacia gum, tara gum, glucomannan, pectin, locust bean gum, guar gum, carrageenan, and tragacanth.

The thickener component may have different origin, i.e. polysaccharide or protein, and include nanocellulose, glucomannan, xanthan gum, gellan gum, diutan gum, welan gum, and pullalun gum, collagen and gelatine.

As specified above, each component of the bioink is essential for i.a. printability. The bioink may comprise a methacrylated gelatin. Methacrylated gelatin (GelMA) is produced through the reaction of gelatin with methacrylic anhydride (MA). A large number of amino groups presenting on the side chains of gelatin are replaced by methacryloyl groups in methacrylic anhydride, forming modified gelatin. GelMA obtains the feature of photocrosslinking because of the presence of methacryloyl groups. GelMA hydrogel is capable of supporting cell behaviors and the biocompatibility and degradation property of gelatin have not been influenced. Furthermore, physical and chemical properties of GelMA hydrogels can be tuned flexibly to meet the requirements of various applications.

By using a methacrylated gelatin, the mechanical stability of a construct produced by bioprinting with the bioink is enhanced. Furthermore, by comprising methacrylated gelatin in the bioink, it is possible to cross-link the constructs, which will further enhance the mechanical stability of the construct.

Additionally, by incorporating thickening agents in the bioink, the rheological properties of the bioinks are improved by increasing the viscosity of the bioink. This will in turn improve the bioprinting process as the bioprinting shape fidelity is improved. In this aspect, bioprinting shape fidelity means that the bioprinted construct will keep its shape upon printing.

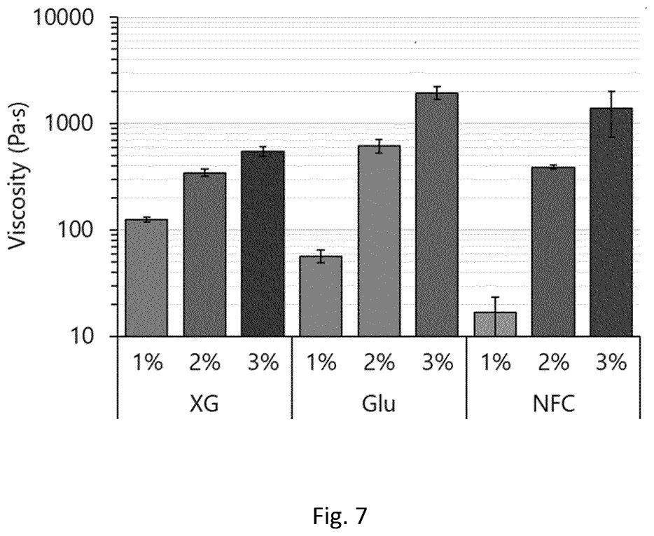

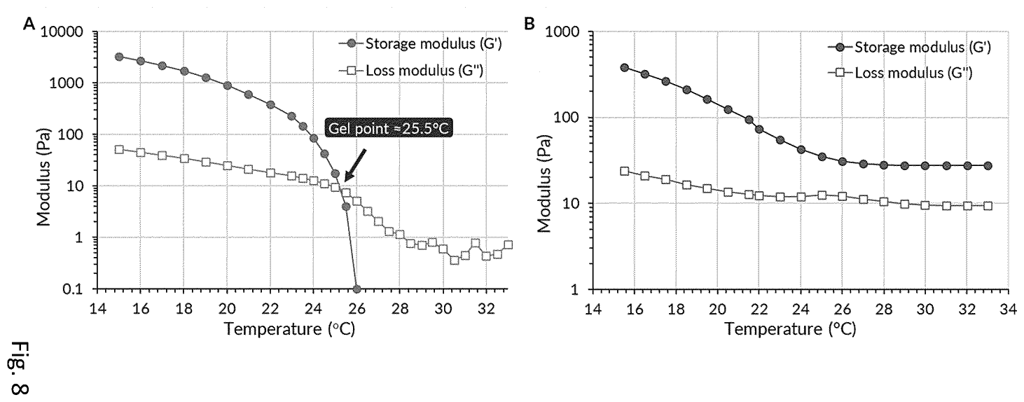

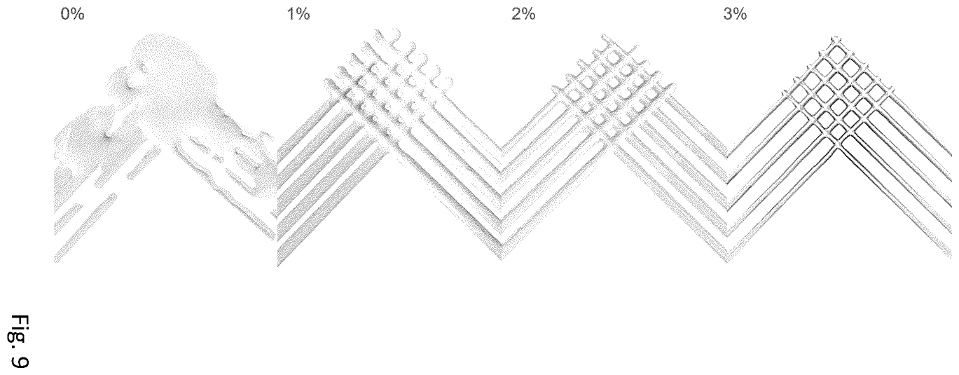

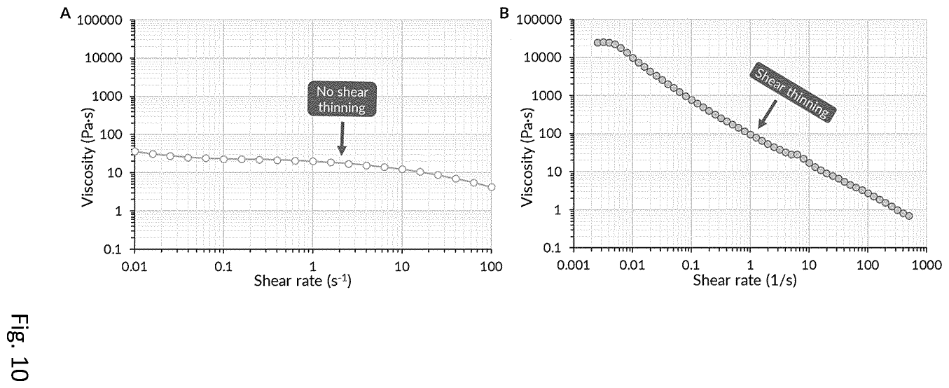

The thickening agents may be natural or synthetic. Preferably, the thickening agent is a natural polysaccharide with neutral effect on cells. Polysaccharide thickeners such as xanthan gum, glucomannan and nanocellulose have been shown to be particularly advantageous thickening agents in bioinks for 3D bioprinting applications. They can modify the viscosity of a bioink (see FIG. 7), shift and increase the printing temperature window (see FIG. 8), and improve the printing resolution of complex multilayered structures (see FIG. 9). The influence on the gelation point leads to less temperature dependence during the bioprinting process. More specifically, this moves the printability temperature to the 20-24° C. range, which is much easier to achieve. The bioprinting can be performed both without temperature-controlled printheads and with temperature controlled printheads for enhanced control. Furthermore, the thickeners may improve the shear thinning properties of the bioinks (see FIG. 10). An increased viscosity provided by the thickeners also appears to protect cells from shear stress during the bioprinting process.

Additionally, the bioink may comprise biopolymers, which may contribute to the crosslinking capacity of the bioink. For instance, alginate may be used as the biopolymer. Alginate will additionally contribute to the ionic crosslinking of the bioink.

The extra-cellular matrix or decellularized matrix components preferably originate from a human and/or animal source, and may be chosen from the group comprising of decellularized extracellular matrices, isolated laminins, glycosaminoglycans such as hyaluronic acid and heparin, proteoglycans, aggrecans, purified molecular proteins such as collagens, elastin, fibrinogen and fibrin and/or purified molecular proteins motifs such as the RGD-motif. In addition, the extra-cellular matrix component may comprise other macromolecules such as exosomes, proteins, ligands, and/or factors isolated/extracted from different animal/human tissues. However, it is also possible to use synthetic extracellular matrix proteins.

The photo initiator is preferably chosen from Lithium phenyl-2,4,6-trimethylbenzoylphosphinate (LAP) or irgacure. However, the skilled person is able to choose a photo initiator that is appropriate for the purpose of the skin tissue model produced.

The cellular additions are added to the bioink in order to obtain a more complex tissue model. It is preferably chosen from one or more of sebocytes, glandular cells, and/or follicle cells. However, the skilled person will be able to identify cellular additions that aid in obtaining a tissue model that is appropriate for the purpose of the produced skin tissue model.

The one or more cell type A is/are chosen from:

(i) Epidermal cells such as keratinocytes, melanocytes, and/or epithelial cells originating from induced pluripotent stem cells, embryonic stem cells, other stem cells, primary cells, and cell lines of human and/or animal origin; or
(ii) Dermal cells such as fibroblasts, endothelial cells, Schwann cells and/or dendritic cells originating from induced pluripotent stem cells, embryonic stem cells, other stem cells, primary cells, and cell lines of human and/or animal origin; or
(iii) Hypodermal cells such as adipose cells, fibroblasts and/or macrophages originating from induced pluripotent stem cells, embryonic stem cells, other stem cells, primary cells, and cell lines of human and/or animal origin.

The at least one cell type A different originates from macro locations, such as a facial location, breast, belly, urethra, oesophagus and/or head, and/or from micro locations, such as papillary dermis or reticular dermis, of the body of healthy, diseased and/or defected human and/or animal sources.

The factor A is a growth factor such as fibroblast growth factor (FGF), epidermal growth factor (EGF), or vascular endothelial growth factor (VEGF) and/or small molecules, macro molecules, and/or proteins such as cytokines, hormones, lipids, carbohydrates or nucleic acids. The Factor A may be chemically attached to, or trapped in, the at least one Bioink A and/or additional bioinks A, and/or incorporated with the at least one cell type A.

The tissue may be bioprinted or dispensed in a manner that produces two or more compartments and/or one or more cellular gradient(s) within the tissue. The tissue may further be bioprinted or dispensed to form a hypodermal compartment, a dermal compartment and/or an epidermal compartment, and optionally a cellular gradient is bioprinted or dispensed within one or more compartment(s). The two or more compartments and/or the cellular gradient(s) may be bioprinted or deposited at the same occasion and/or at one or more later occasions. An epidermal compartment corresponds to epidermis which is the part of skin tissue that is outwards and facing the environment, the hypodermal compartment corresponds to hypodermis which is the lower part of skin tissue facing other internal tissues, and the dermal compartment corresponds to derma, which is a part of skin tissue between the epidermis and hypodermis. FIG. 2 shows an embodiment where the skin tissue model has been bioprinted with an epidermal compartment A, and a gradient, B and C, within the dermal compartment. However, the design could be the same for all three compartments, adjusting the cell type(s) and bioink composition in compartment C to represent the hypodermis. The different compartments could thus represent the epidermal (A), dermal (B) and hypodermal (C) compartments of skin. In addition, the distribution of biomolecules within the different layers of a skin tissue model and/or layering of different bioinks within the composition is illustrated in FIG. 2, by the gradient of cells as indicated therein.

The cell suspension A disclosed above may be added to the bioprinted dermal compartment, and/or to the bioprinted hypodermal compartment, and/or to the bioprinted epidermal compartment.

The produced skin tissue model may further be subject to a culturing method wherein the skin tissue model is cultured

(i) by being submerged in medium;
(ii) in a flow device to mimic a vascular system; and/or
(iii) at an air-liquid interface.

A combination of one or several culturing methods may be used for the same skin tissue model, either simultaneously and/or sequentially. When using an air-liquid interface, it is preferable to arrange the skin tissue so that hypodermal compartment is in contact with the liquid medium, and the epidermal compartment is exposed to air. A flow device may be used for the culturing to mimic distribution of nutrients via a vascular system and/or to mimic sporadically flowing of body liquids over internal skin linings, such as in the case of for instance the urethra. In general, the culturing of the skin tissue models according to the present disclosure should be performed in an appropriate medium, as a skilled person is readily able to determine based on the cells that are used in the skin tissue model. Furthermore, standard culture conditions should be applied, such as a temperature of about 37° C., about 5% CO₂, and a relative humidity of about 95%. Culturing conditions and culture media are part of the common general knowledge for the skilled person.

It is also possible to add the Factor A continuously to the produced skin tissue model after the bioprinting thereof and during the cultivation with any of the above-mentioned culturing methods. This may lead to a maturation of the skin tissue model, depending on the Factor A being used. It may alternatively lead to a development of the skin tissue model that is appropriate for the study to be conducted on said skin tissue model, such as inducing a specific condition.

The method of producing a skin tissue model according the present invention thus enables layering of the components to mimic the native distribution of cells, biomaterials and biomolecules in the skin. In addition, this enables to create niche environments within the skin. Further, by culturing the skin tissue model with an appropriate culture method, native environments may be mimicked. Thus, the method of the present invention allows the user to generate complex 3D structures within the tissue model with many types of 3D bioprinting technologies, and many types of application of the skin tissue model produced.

According to a second aspect, the present disclosure provides for a 3D bioprinted skin tissue model, comprising at least one bioink A, at least one cell type A, at least one factor A. The bioink A comprises at least one biopolymer, a thickener, at least one extra-cellular matrix or a decellularized matrix, and optionally a photo initiator and/or cellular additions. The at least one cell type A is an epidermal, dermal and/or hypodermal cell or cell line of human and/or animal origin, said cells optionally being primary cells, immortalized and iPSC- or ESC-derived. The at least one factor A is a protein or molecule that will stimulate altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition. The components and quantities comprised in the 3D bioprinted skin tissue model are in general the same as those disclosed above for the method of the present invention.

The biopolymer is chosen from biopolymer such as collagen, methacrylate collagen (ColMA), gelatine, methacrylate gelatine (GelMA), cellulose, nano-fibrillar cellulose, alginate or chitosan. The biopolymer can be a polysaccharide derived from botanical sources such as acacia gum, tara gum, glucomannan, pectin, locust bean gum, guar gum, carrageenan, and tragacanth.

According to one embodiment of the second aspect, the skin tissue model comprises an additional cell suspension A, said cell suspension A comprises of cell relevant medium and/or materials synthetically derived or derived from bacteria, plants and/or animals, such as gelatine methacrylate, collagen, collagen methacrylate, alginate or cellulose, optionally a thickener, a cell type A, factors specific to cell type A which are proteins or molecules that will stimulate altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition, optionally a photo initiator, optionally extracellular matrix proteins.

The one or more cell type A is/are chosen from

(i) Epidermal cells such as keratinocytes, melanocytes, and/or epithelial cells originating from induced pluripotent stem cells, embryonic stem cells, other stem cells, primary cells, and cell lines of human and/or animal origin; or
(ii) Dermal cells such as fibroblasts, endothelial cells, Schwann cells and/or dendritic cells originating from induced pluripotent stem cells, embryonic stem cells, other stem cells, primary cells, and cell lines of human and/or animal origin; or
(iii) Hypodermal cells such as adipose cells, fibroblasts and/or macrophages originating from induced pluripotent stem cells, embryonic stem cells, other stem cells, primary cells, and cell lines of human and/or animal origin.

The model may comprise at least one compartment, representing a hypodermal, a dermal and/or an epidermal compartment. The model may further comprise two or more compartments representing a biological gradient corresponding to a hypodermal, a dermal and/or an epidermal compartment; and may optionally comprise a biological gradient within one or more of said compartments.

According to a third aspect there is provided for the use of the 3D bioprinted skin tissue model according to the above in one or more of:

(i) Developmental biology in order to gain understanding of cellular activities within a 3D environment such as cellular distribution, migration, proliferation, matrix production, interactions with other cells and the surrounding environment, etc.; and/or
(ii) Compound testing for cosmetic and skin care product evaluation, toxicity studies, irritant studies, allergen testing, metabolism studies, tissue and/or cellular rejuvenation investigations, photosensitivity testing, drug and/or molecular compound absorption testing, cellular differentiation/maturation, spheroid differentiation/maturation, organoid differentiation/maturation, etc.; and/or
(iii) Tissue regeneration and rejuvenation applications such as tissue remodelling, cellular proliferation, cellular metabolism, cellular differentiation/maturation, cell-cell interaction, cell-matrix interaction, cellular crosstalk/signalling, vascularization, etc.; and/or
(iv) Pharmaceutical applications for drug discovery, target validation, allergen studies, toxicity studies, metabolism studies, cellular differentiation/maturation, spheroid differentiation/maturation, organoid differentiation/maturation, etc.; and/or
(v) Medical device evaluation and development, toxicity studies, allergen studies etc. for devices in contact with internal and/or external skin linings; and/or
(vi) Stem cell research with focus on cellular differentiation and maturation as dispersed cells, spheroids, organoids, etc.

It may thus relate to cosmetic compound evaluation and/or discoveries, evaluation of medical devices, skin care compound evaluation and/or discoveries, pharmaceutical evaluations and/or discoveries, regenerative medicine investigations, tissue and/or cellular rejuvenation investigations, photosensitivity testing, drug and/or molecular compound absorption testing, tissue engineering developments, toxicology studies, irritant studies, allergen testing and skin physiology and/or pathology.

The use of the 3D bioprinted skin tissue model according to the present invention may be performed in applications relating to both internal skin linings, such as the oesophagus and the urethra, and external skin linings such as the skin.

### Example of Production Procedure

Below is an example of producing the skin tissue model with a bioink A, cell type A and an additional cell type A and one factor A:

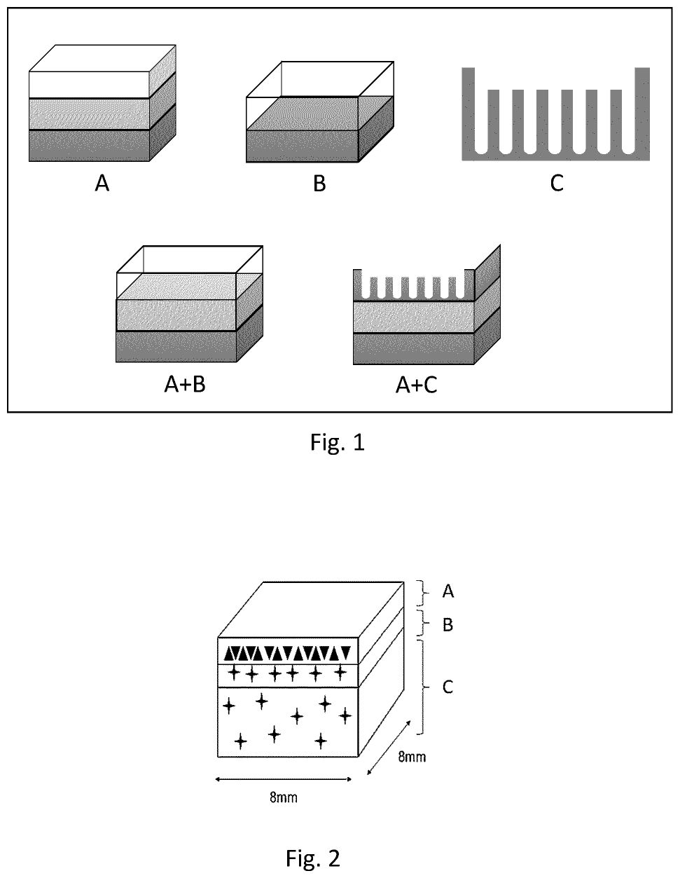

Design of model (s): To design the model any type of 3D-modeling software can be utilized to either modify a scanned model, such as a CT- or MR-scan, or to create a model from scratch. When creating models from scratch different types of compartments can be used as building blocks. FIG. 1 exemplifies 3 types of compartment types and two examples of how these can be combined to create a model.
A. Block Compartments. These blocks can have different dimensions, and either be stacked and/or placed within each other to form different compartments within the model. Using robust bioink formulations able to retain the shape of the blocks during the print blocks can be utilized to form one or several micro compartments within or throughout a model. They can also be used to form gradients by enabling deposition of different bioink A, cell type A and/or factor (s) A in different layers/levels of the model. These blocks could also be of cylindrical shape and/or of any polyhedron shape.
B. Brim Compartments. A brim is a wall structure that can be shaped on top of a block model to form one or several wells or within a model to separate compartments or parts of the model, for example. When placed on top of a model it can serve as a container to hold less viscous bioink (s), with or without cells such as keratinocytes and/or melanocytes, and/or factors (as exemplified in this disclosure). One such model of A+B is exemplified in FIG. 1.
C. Rigid Compartments. Rigid compartments create pits that could be used as groves and/or to form aggregation within or on top of models, for example. When placed on-top or within a block model the formed pits could be utilized as aggregation point for spheroids, organoids, glands and/or follicle formation by guiding deposit bioink (s) with cell (s) and/or additional factor (s) together in the bottom of the pits. One such model of A+C is exemplified in FIG. 1.

The set and finalized design is the blueprint of the model. FIG. 2 exemplifies a blueprint of a model consisting of 3 block compartments where the blocks are utilized to form a gradient of fibroblasts within the dermis. Here the base of the blocks is sat to 8×8 mm. Compartment A in FIG. 2 represents the epidermis of skin and is printed as one solid layer with Bioink A, Cell Type A, 0.5×10⁶ to 50×10⁶ cells per mL of bioink A, and 80% rectilinear infill. Compartment B in FIG. 2 represents in this example the papillary dermis of skin and is printed as one porous layer with Bioink A, additional Cell Type A, 5×10⁶ to 10×10⁶ cells per mL of bioink A, and 20% rectilinear infill. Compartment C in FIG. 2 represents in this example the reticular dermis of skin and is printed as two or three porous layers with Bioink A, additional Cell Type A, 0.5×10⁶ to 5×10⁶ cells per mL of bioink A, and 10% grid infill. Such a model benefit of being printed up-side down, starting with the solid layer A, then print the porous structure B and lastly print the most porous structure C on top. The porous structure allows for faster medium diffusion and efficient use of materials and require stable bioinks to keep shape while printed.

The height of block A, B and C is sat in consideration to which nozzle or needle that the model is intended to be printed with. For a 22 G nozzle, which has an inner diameter of 0.41 mm, a layer height of 0.4-0.5 mm could be used for each intended layer. To create a g-code of the blueprint each block, A, B and C, are saved separately as a 3D file, for example in stl format, and then imported into a slicing software (if not this is possible in the same program as the 3D files where created in). In the slicing software align the blocks on top of each other and assign block A printhead 1, block B printhead 2 and block C print head 3. Set infill pattern and density for each block, as described above, and export the g-code file. If needed, if you cannot adjust the speed or extrusion rate on your bioprinter, also set these parameters in the slicing program. For speed 5-10 mm/s are good starting points but will need to be adjusted to the features, such as viscosity and temperature, of Bioink A. Parameters such as viscosity, temperature and shear thinning of Bioink A as well as choice of nozzle or needle will influence the extrusion rate or pressure needed for the print.

Bioprinting of model (s): Make sure g-code is working properly and that all materials needed, such as nozzles, cartridges and luer lock adaptors, are sterilized prior to print. Pre-warm Bioink A, 0.5-1.5 mL per block compartment to print 24 replicates. I.e. a full 24 well plate. Prepare the 3 cell suspensions by firstly detach and count cell type A and additional cell type A according to protocol of the cells. Secondly resuspend the right number of cells to achieve wanted cell concentration in a nutritious solution with a total volume of 100 μL cell suspension per 1 mL of bioink A. Distribute the bioink into 3 different syringes of 1-3 mL volume to be mixed with right cell type A and/or right concentration of additional cell type A. Mix right cell suspension with right amount of bioink, using for example two syringes connected with a Female/Female luer lock adaptor, and transfer the bioink with embedded cells to a cartridge. Mount the cartridges into assigned printhead, with bioink A with cell type A for block A in printhead 1, bioink A with additional cell type A for block B in printhead 2 and bioink A with additional cell type A for block C in printhead 3, as set in the blueprint. Flip bioprinted models after curing to have epidermis facing up, as the models in this example was printed up-side down. Not needed if the bioprinted model (s) already are facing the right direction.

Curing of bioprinted model: Curing is the process of chemically and/or physically change and/or activate features of the Bioink A to make the bioprinted model crosslink and become a stable construct. Curing of the bioprinted model (s) can either be performed during the print, either layer by layer or after completion of one construct, and/or after all replicates of the models are printed and the printing process is finalized. Curing could be done using photocuring, for example wave lengths such as 365 or 405 nm, a Factor A, such as an enzyme or a protein, and/or ions. In this example Bioink A can be photocured for 15-45 seconds at a distance of 3-5 cm above the model and/or ionically crosslinked for 3-5 minutes with a cell neutral ionic solution. After curing samples are incubated with cell culture medium at standard culture conditions (37° C., 5% CO₂ and 95% relative humidity), or washed to remove excess ions and then incubated, with Factor A, for example thrombin, for 0.01-48 h to activate native features in Bioink A, cell type A and/or additional cell type A.

3D culture and analysis of model (s): The can upon completion be cultured and analysed as needed to fulfil its purpose. To form a complete skin tissue model the bioprinted constructs are recommended to be cultured submerged in media for at least 2 days in proliferative cell culture conditions prior to initiating differentiation (maturation) of the model, by for example altering the medium composition or raising the bioprinted models to air-liquid interface.

Examples of analysis that can be performed during culture of the bioprinted skin tissue model (s) is viability analysis by staining samples or a part of a construct for viable respectively dead cells. This analysis tells, in addition to how well the cells are doing in the bioprinted construct, morphological development, mobility and spatial arrangement of the cells. For example, in FIG. 3 viability analysis is used to show how the dermal fibroblasts in a bioprinted skin tissue model can differ depending on culture conditions. FIG. 3A are fibroblasts cultured in a 3D bioprinted skin tissue model with one Bioink A without an epidermis while FIG. 3B show fibroblasts cultured in a 3D bioprinted skin tissue model with the same Bioink A with an epidermis. Where only the fibroblasts in FIG. 3B develops the elongated morphology typical for dermal fibroblasts. FIG. 3C show how viability analysis could be used to visualize the spatial arrangement of the bioprinted model, with the brighter cells representing the epidermis and less intense cells being the dermal fibroblasts their deposition within the construct is seen clearly.

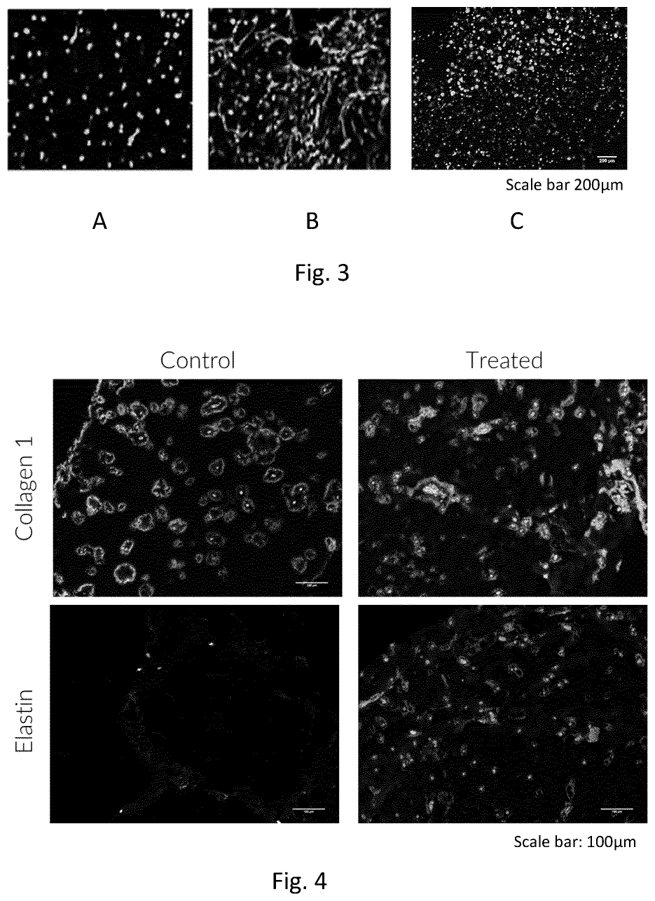

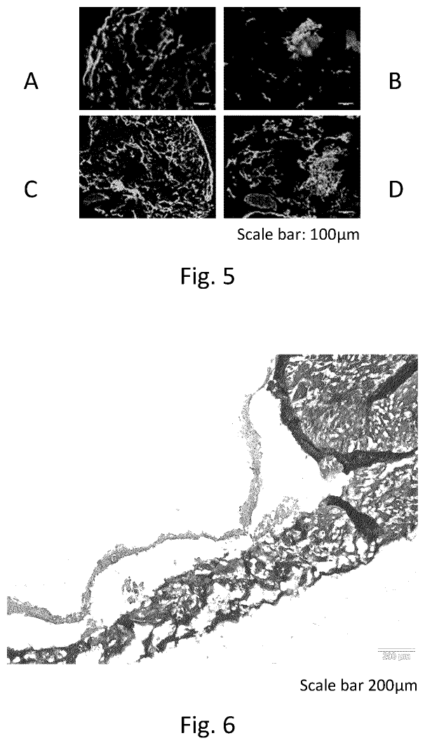

Other examples of analysis that can be performed on the bioprinted skin tissue model (s) are snap freezing of samples for qPCR analysis and fixation of constructs for immunohistology or immunofluorescence staining, among other analysis methods. FIG. 4 show an example of how immunofluorescence staining can be utilized to evaluate the protein expression profiles in different treatments of the bioprinted skin tissue models. In FIG. 4 the elastin expression in the dermis can be seen to sharply increase when treated with a specific factor A and in FIG. 5 it is shown how the collagen type 1 expression develops over time, both in a treated and a non-treated sample. FIG. 6 show an example of how immunohistology can be utilized to analyse model maturation of cross sections of the bioprinted samples, here the epidermis can be noted to form on top of a bioprinted dermis Bioprinted with a bioink A and cell type A.

1. A method of producing a skin tissue model in an automated manner, comprising the steps of:
(a) providing at least one bioink A;
(b) providing at least one cell type A;
(c) providing at least one factor A;
(d) mixing the components provided in steps (a)-(c), and optionally other components, in such proportions that allows printability for the mixture, and that provides a viable setting for the at least one cell type A;
(e) bioprinting and/or dispensing the resulting mixture in an automated and reproducible manner, whereby a tissue model is formed, said tissue being characterized as a skin tissue;
wherein the bioink A comprises at least one biopolymer, a thickener, at least one extra-cellular matrix or a decellularized matrix component, and optionally a photo initiator and/or cellular additions, such as sebocytes, glandular cells and/or follicle cells;
wherein the thickener is a polysaccharide-based substance, such as nanocellulose, glucomannan, xanthan gum, gellan gum, diutan gum, welan gum or pullalun gum, or a protein-based substance, such as collagen or gelatin, that modulates the viscosity of the bioink A;
the at least one cell type A is an epidermal, dermal and/or hypodermal cell or cell line of human and/or animal origin, said cells optionally being primary cells, immortalized and iPSC- or ESC-derived; and
the at least one factor A is a growth factor, such as fibroblast growth factor (FGF), epidermal growth factor (EGF), or vascular endothelial growth factor (VEGF), and/or small molecules, macro molecules, and/or proteins such as cytokines, hormones, lipids, carbohydrates or nucleic acids, that stimulates altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition.

2. The method according to claim 1, wherein:
the bioink A comprises (based on total weight of the bioink) 2-15% w/w, preferably 2-10% w/w, of at least one biopolymer, 0.5-3% w/w of thickener, 0.1-2% w/w of at least one extra-cellular matrix or a decellularized matrix component, and optionally 0.05-1% w/w of a photo initiator and/or 1×10²-1×10⁷ cellular additions per ml;
the at least one cell type A is used in quantities of 1×10³-10×10⁷ cells per 1 mL Bioink and/or 1×10³-10×10⁵ cells per 1 cm²; and/or
the at least one factor A is used in quantities of 1×10⁻⁹-1×10⁻³ molar for growth factors, and 1×10⁻⁶-1×10⁻¹ molar and/or 1-1000 mg/mL of other factors.

3. The method according to claim 1 or 2, wherein at least one additional cell type A, at least one additional bioink A, and at least one additional Factor A is provided, wherein the two or more bioinks are formulated so that bioink A supports one cell type A and the additional bioink(s) A support(s) a second or further cell type A.

4. The method according to any of claims 1-3, further comprising a step (f) of providing a cell suspension A, and applying said cell suspension A to the tissue formed in step (e), wherein the cell suspension A comprises cell relevant medium and/or materials synthetically derived or derived from bacteria, plants and/or animals, such as gelatine methacrylate, collagen, collagen methacrylate, alginate or cellulose,
optionally a thickener,
a cell type A,
factors specific to cell type A which are proteins or molecules that will stimulate altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition,
optionally a photo initiator,
optionally extracellular matrix proteins.

5. The method according to any of the claims 1-4, wherein the at least one biopolymer is chosen from the group comprising a nanocellulose or nanofibrillar cellulose, a gelatine, such as gelatine methacrylate, alginate, acacia gum, tara gum, glucomannan, pectin, locust bean gum, guar gum, carrageenan, and tragacanth.

6. The method according to any of claims 1-5, wherein the thickener is a polysaccharide-based substance, such as nanocellulose, glucomannan, xanthan gum, gellan gum, diutan gum, welan gum or pullalun gum, or a protein-based substance, such as collagen or gelatin;

7. The method according to any of claims 1-6, wherein the extra-cellular matrix or decellularized matrix components originate from a human or animal source, and may be chosen from the group comprising of glycosaminoglycans, collagens, elastin, proteoglycans, aggrecans, isolated laminins, glycol-amino-glycans such as hyaluronic acid and heparin, purified molecular proteins such as fibrinogen and fibrin and/or purified molecular proteins motifs such as the RGD-motif.

8. The method according to any of the claims 1-7, wherein the photo initiator is chosen from Lithium phenyl-2,4,6-trimethylbenzoylphosphinate (LAP) or irgacure.

9. The method according to any of the claims 1-8, wherein the cellular addition is chosen from one or more of sebocytes, glandular cells, and/or follicle cells.

10. The method according to any of the claims 1-9, wherein the one or more cell type A is/are chosen from:
(i) Epidermal cells such as keratinocytes, melanocytes, and/or epithelial cells originating from induced pluripotent stem cells, embryonic stem cells, other stem cells, primary cells, and cell lines of human and/or animal origin; or
(ii) Dermal cells such as fibroblasts, endothelial cells, Schwann cells and/or dendritic cells originating from induced pluripotent stem cells, embryonic stem cells, other stem cells, primary cells, and cell lines of human and/or animal origin; or
(iii) Hypodermal cells such as adipose cells, fibroblasts and/or macrophages originating from induced pluripotent stem cells, embryonic stem cells, other stem cells, primary cells, and cell lines of human and/or animal origin.

11. The method according to any of the claims 1-10, wherein the at least one cell type A originates from macro locations, such as a facial location, breast, belly, urethra, oesophagus and/or head, and/or from micro locations, such as papillary dermis or reticular dermis, of the body of healthy, diseased and/or defected human and/or animal sources.

12. The method according to any of the preceding claims, wherein step (e) is performed using an extrusion, syringe or ink-jet based bioprinting device.

13. The method according to any of the preceding claims, wherein the tissue is bioprinted or dispensed in a manner that produces two or more compartments and/or one or more cellular gradient(s) within the tissue.

14. The method according to claim 13, wherein the tissue is bioprinted or dispensed to form a hypodermal compartment, a dermal compartment and/or an epidermal compartment, and optionally wherein a cellular gradient is bioprinted or dispensed within one or more compartment(s).

15. The method according to any of the claims 13-14, wherein the two or more compartments and/or the cellular gradient(s) are bioprinted or deposited at the same occasion and/or at one or more later occasions.

16. The method according to any of the preceding claims, wherein the Factor A is chemically attached to, or trapped in, the at least one Bioink A and/or additional bioinks A, and/or incorporated with the at least one cell type A.

17. The method according to any of the preceding claims, wherein the produced skin tissue model is further subject to a culturing method wherein the skin tissue model is cultured
(i) by being submerged in medium;
(ii) in a flow device to mimic a vascular system; and/or
(iii) at an air-liquid interface.

18. The method according to claim 17, wherein a combination of one or several culturing methods is used for the same skin tissue model, either simultaneously and/or sequentially.

19. A 3D bioprinted skin tissue model, comprising
i. at least one bioink A
ii. at least one cell type A
iii. at least one factor A
wherein the bioink A comprises at least one biopolymer, a thickener, at least one extra-cellular matrix or a decellularized matrix, and optionally a photo initiator and/or cellular additions;
the at least one cell type A is an epidermal, dermal and/or hypodermal cell or cell line of human and/or animal origin, said cells optionally being primary cells, immortalized and iPSC- or ESC-derived;
wherein the thickener is a polysaccharide-based substance, such as nanocellulose, glucomannan, xanthan gum, gellan gum, diutan gum, welan gum or pullalun gum, or a protein-based substance, such as collagen or gelatin; and
the at least one factor A is a growth factor, such as fibroblast growth factor (FGF), epidermal growth factor (EGF), or vascular endothelial growth factor (VEGF), and/or small molecules, macro molecules, and/or proteins such as cytokines, hormones, lipids, carbohydrates or nucleic acids that stimulates altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition.

20. The 3D bioprinted skin tissue model according to claim 19, wherein:
the bioink A comprises (based on total weight of the bioink) 2-15% w/w, preferably 2-10% w/w, of at least one biopolymer, 0.5-3% w/w of thickener, 0.1-2% w/w of at least one extra-cellular matrix or a decellularized matrix component, and optionally 0.05-1% w/w of a photo initiator and/or 1×10²-1×10⁷ cellular additions per ml;
the at least one cell type A is used in quantities of 1×10³-10×10⁷ cells per 1 mL Bioink and/or 1×10³-10×10⁵ cells per 1 cm²; and/or
the at least one factor A is used in quantities of 1×10⁻⁹-1×10⁻³ molar for growth factors, and 1×10⁻⁶-1×10⁻¹ molar and/or 1-1000 mg/mL of other factors.

21. The 3D bioprinted skin tissue model according to any of the claims 19-20, wherein the at least one biopolymer is chosen from a nanocellulose, or nanofibrillar cellulose, or a gelatine, such as gelatine methacrylate.

22. The 3D bioprinted skin tissue model according to any of the claims 19-21, further comprising an additional cell suspension A, said cell suspension A comprises of cell relevant medium and/or materials synthetically derived or derived from bacteria, plants and/or animals, such as gelatine methacrylate, collagen, collagen methacrylate, alginate or cellulose, optionally a thickener, a cell type A, factors specific to cell type A which are proteins or molecules that will stimulate altered or abnormal metabolism of cell type A, said factor A being specific to epidermal, dermal and/or hypodermal cells and promoting cell proliferation, cellular repair, dermal vascularization, skin tissue maturation and/or other cellular stimuli such as motility and/or inhibition, optionally a photo initiator, optionally extracellular matrix proteins.

23. The 3D bioprinted skin tissue model according to any of the claims 19-22, wherein the one or more cell type A is/are chosen from
(i) Epidermal cells such as keratinocytes, melanocytes, and/or epithelial cells originating from induced pluripotent stem cells, embryonic stem cells, other stem cells, primary cells, and cell lines of human and/or animal origin; or
(ii) Dermal cells such as fibroblasts, endothelial cells, Schwann cells and/or dendritic cells originating from induced pluripotent stem cells, embryonic stem cells, other stem cells, primary cells, and cell lines of human and/or animal origin; or
(iii) Hypodermal cells such as adipose cells, fibroblasts and/or macrophages originating from induced pluripotent stem cells, embryonic stem cells, other stem cells, primary cells, and cell lines of human and/or animal origin.

24. The 3D bioprinted skin tissue model according to any of the claims 19-23, wherein the model comprises at least one compartment, representing a hypodermal, a dermal and/or an epidermal compartment.

25. The 3D bioprinted skin tissue model according to any of the claims 19-24, wherein the model comprises two or more compartments representing a biological gradient corresponding to a hypodermal, a dermal and/or an epidermal compartment; and optionally comprising a biological gradient within one or more of said compartments.

26. Use of the 3D bioprinted skin tissue model according to any of the claims 19-25, in one or more of:
(i) Developmental biology in order to gain understanding of cellular activities within a 3D environment such as cellular distribution, migration, proliferation, matrix production, interactions with other cells and the surrounding environment, etc.; and/or
(ii) Compound testing for cosmetic and skin care product evaluation, toxicity studies, irritant studies, allergen testing, metabolism studies, tissue and/or cellular rejuvenation investigations, photosensitivity testing, drug and/or molecular compound absorption testing, cellular differentiation/maturation, spheroid differentiation/maturation, organoid differentiation/maturation, etc.; and/or
(iii) Tissue regeneration and rejuvenation applications such as tissue remodelling, cellular proliferation, cellular metabolism, cellular differentiation/maturation, cell-cell interaction, cell-matrix interaction, cellular crosstalk/signalling, vascularization, etc.; and/or
(iv) Pharmaceutical applications for drug discovery, target validation, allergen studies, toxicity studies, metabolism studies, cellular differentiation/maturation, spheroid differentiation/maturation, organoid differentiation/maturation, etc.; and/or
(v) Medical device evaluation and development, toxicity studies, allergen studies etc. for devices in contact with internal and/or external skin linings; and/or
(vi) Stem cell research with focus on cellular differentiation and maturation as dispersed cells, spheroids, organoids, etc.

27. Use of the 3D bioprinted skin tissue model according to claim 26 in applications relating to both internal and external skin linings such as the skin, oesophagus and urethra.
